(19) United States
(12) Patent Application Publication
(10) Pub. No.: US 2002/0004050 A1
(43) Pub. Date: Jan. 10, 2002

(54) Vaccines

(75) Inventors: Robert Llewelyn Clancy, Newcastle (AU); Margaret Dunkley, Elermore Vale (AU)

Correspondence Address: PENNIE & EDMONDS LLP, 1667 K Street, N.W., Washington, DC, 20006, US

(73) Assignee: Auspharm International Limited

(21) Appl. No.: 09/731,878

(22) Filed: Dec. 8, 2000

Related U.S. Application Data
(63) Continuation of application No. 09/027,826, filed on Feb. 20, 1998, now abandoned.

(30) Foreign Application Priority Data
Aug. 22, 1996 (GB) PCT/GB99/02048

(51) Int. Cl. A61K 39/295; A61K 39/12

(52) U.S. Cl. 424/204.1; 424/201.1

(57) ABSTRACT

Vaccines useful in providing protection against lower respiratory tract infections are provided as well as methods of immunising a subject.

The present invention relates to novel vaccine compositions, as well as the use of one or-more antigens derived from H.influenzae in the manufacture of an influenza vaccine and to methods of vaccination against influenza virus infection.

In patients with chronic obstructive lung disease (COLD) morbidity and mortality are associated with acute episodes of bronchitis. The upper respiratory tract of such patients is commonly colonised with Haemophilus influenzae and/or Streptococcus pneumoniae.

Influenza is a common disease which affects large numbers of people every year. It is particularly life threatening in the elderly (indeed 85% of deaths associated with the disease occur in patients over 75) the very young and those whose immune systems are already weakened through the effects of another infection.

In addition, interaction between the influenza virus and H. influenzae in particular, can increase infectivity of both organisms. It is thought that the virus reduces physical aspects of a host's defence, e.g. by damaging cilia, which will lead to increased bacterial colonisation. Meanwhile, enzymes produced by, inter alia, H. influenzae and the inflamed bronchus (Scheiblauer et al, J. Infect. Dis. 166: 783-791 (1992); Keikkinen et al, Options for the Control of Influenza II, Edited by Hannonn, C., Kendal, A. P., Klenk, H. D., Ruben, F. L., Amsterdam, Elsevier: 431-434 (1993)) cleave the distal end of haemagglutinin (Walker, J. A., Kawaoka, Y., J. Gen. Virol. 74: 311-314 (1993)) which in turn increases virus infectivity. The result of these effects is a vicious cycle of interaction between bacteria and virus.

We have now demonstrated that infection with the influenza virus does indeed lead to very large increases in bacterial colonisation of the lungs, which in turn can lead to death. Based on this, therefore, we have found that protection against, influenza can be afforded by vaccines against bacterial infections, eg H. influenzae vaccines. EP-A-0225329 describes such vaccines.

In addition, simultaneous vaccination against influenza virus and H. influenzae may provide synergistic levels of protection against lower respiratory tract infection.

Thus, in a first aspect, the present invention provides a vaccine for use in the prevention of influenza which comprises at least one antigen derived from H.influenzae.

Such a vaccine will prevent the large scale bacterial population growth associated with influenza virus infection and hence will protect a subject against the detrimental effects of the virus infection associated with the bacterial growth.

In addition, since it is the influenza virus infection itself which triggers the bacterial proliferation, a combination vaccine comprising at least one antigen from both H.influenzae and influenza virus will be particularly effective in protecting a subject.

Thus, in a second aspect, the present invention provides a vaccine comprising:

(i) at least one antigen derived from H. influenzae; and

(ii) at least one antigen derived from influenza virus.

Such a combination vaccine will provide effective protection against influenza and may also result in enhanced levels of protection against lower respiratory tract infection per se. Suitable influenza virus vaccines are described in, for example, WO-A-9116073.

The H. influenzae antigen(s) can be derived from e.g. live bacteria or killed bacteria. The antigen(s) can be isolated or presented in the form of whole cells. The influenza virus antigen(s) can be provided in the form of irradiated virus particles.

It is also possible to administer the individual components separately to induce immunity against both particular bacteria, e.g. H. influenzae, and influenza virus. Therefore, in a third aspect, the present invention provides a vaccine comprising:

(i) at least one antigen derived from H. influenzae; and

(ii) at least one antigen derived from influenza virus;

for simultaneous, separate or sequential use.

Suitably, in the above-noted combination vaccines the vaccines of EP-A-0225329 and WO-A-9116073 can be used.

Another advantage of the vaccine compositions of the invention is that they produce a mucosal immune response which is particularly desired for combatting lower respiratory tract infection.

In preferred embodiments of the above aspects, the vaccines can be administered as nasal drops, or as “drops under the tongue”, i.e. as a sub-lingual preparation.

In other embodiments, the H.influenzae vaccine or vaccine component is an enteric vaccine. This may be in the form of tablets, especially enteric coated tablets, granules, capsules or dragees for oral administration, or provided e.g. as suppositories for rectal administration. The dosage unit form may contain from approximately 10⁹ bacteria to approximately 10¹³ bacteria, preferably from approximately 10¹⁰ bacteria to approximately 10¹² bacteria, together with suitable carriers or organic of inorganic nature.

Suitable carriers are especially fillers, such as sugars, for example lactose, saccharose, mannitol or sorbitol, cellulose preparations and/or calcium phosphates, for example tricalcium phosphate or calcium hydrogen phosphate, amino acids, for example glycine, also binders, such as starch pastes using, for example, corn wheat, rice or potato starch, gelatine, tragacanth, methylcellulose, hydroxypropylmethylcellulose, sodium carboxymethylcellulose and polyvinylpyrrolidone, and/or, if desired, disintegrators, such as the above-mentioned starches and also carboxymethyl starch, cross-linked polyvinylpyrrolidone, agar, alginic acid or a salt thereof, such as sodium alginate. Adjuncts are especially flow-regulating agents and lubricants, for example, silica, talc, stearic acid or salts thereof, such as magnesium stearate or calcium stearate, and/or polyethylene glycol, further choleretic agents, e.g. sodium taurocholate, sodium tauroglycocholate or ox bile. Dragee cores are provided with suitable coatings that may be resistant to gastric juices, there being used, inter alia, concentrated sugar solutions which may contain gum arabic, talc, polyvinylpyrrolidone, polyethylene glycol and/or titanium dioxide, lacquer solutions in suitable organic solvents or solvent mixtures, or, for the production of coatings that are resistant to gastric juices, solutions of suitable cellulose preparations, such as acetylcellulose phthalate or hydroxypropylmethylcellulose phthalate. Dyes or pigments can be added to the tablets or dragee coatings, for example for identification purposes or to indicate different doses of active ingredient.

The bacteria may be cultured and then killed, lyophilised and/or dried, mixed with carriers, fillers and adjuncts, using methods well known in the art. For instance, the bacteria may be grown on plates, e.g. agar plates containing various nutrients, for example blood or “chocolate” agar plates, or in suspension in fermentation broth containing nutrients in dissolved form, e.g. milk hydrolysates, lactalbumin hydrolysates, corn steep liquors, glucose, starches, tryptic soy broth and the like, and growth stimulating substances, e.g. hormones and coenzymes, also in the form of serum dilutions, for example of horse serum or foetal calf serum. The cultures must be kept under strictly aseptic conditions, and optionally small amounts of antibiotics, e.g. bacitracin, are added to prevent overgrow by unwanted bacteria.

The organisms are killed, for example with formalin, phenol or ether, optionally homogenised and washed extensively. Sterility of the killed bacteria has to be tested carefully, e.g. by inoculation into a medium or an agar plate known to allow rapid growth of viable bacteria. Pharmaceutical preparations for oral or rectal administration are obtained from killed bacteria by conventional lyophilising, drying, mixing, granulating and/or confectioning processes under sterile conditions.

The vaccines of WO-A-9116073 are particularly effective in inducing mucosal immunity, and as such represent the preferred form for presenting the viral antigen(s). These vaccine compositions can be administered orally and thus oral vaccine combinations represent a further preferred embodiment of the present invention.

In a fourth aspect, the present invention provides a vaccine composition comprising:

(i) an enteral non-adjuvenated non-bacterial vaccine comprising killed H. influenzae and optionally a pharmaceutical carrier; and

(ii) a vaccine comprising at least one antigen derived from influenza virus surface-associated with red blood cells or derivatives thereof;

for simultaneous, separate or sequential use.

Thus, in this aspect, the individual vaccine components of the composition can be delivered as a single composition. Alternatively, the individual components could be presented separately for simultaneous or sequential administration.

This aspect of the invention is not limited to the use of whole red blood cells in the vaccine, because derivatives thereof, such as ghosts or membrane preparations, including parts of membranes, can also yield the desired enhanced mucosal immunogenicity. One observation has been that the use of red blood cells provides extremely uniform size particles within the optimal 5 to 10 μm range optimally taken up by Peyers patches. Thus, by the use of the invention, the influenza antigen can be effectively targeted to the Peyers patches, the “mucosal motor” for the activation of the common mucosal system.

The surface-association of the antigen(s) with the red blood cell or derivative may take place through adsorption or binding via liganding or other chemical modification. One alternative is to bind the antigen to a lectin or antibody (fragment) having specificity for red blood cell markers. Preferably, however, the association takes place through the interaction of an indigenous (i.e. naturally present) receptor on the red blood cell, the receptor having specificity either for the antigen itself or for a linking or haptenic group attached to the antigen.

For instance, the influenza haemagglutinin glycoprotein (HA) binds avidly to a surface receptor of chicken red blood cells (CRBC). Influenza preparations, either live attenuated or inactivated, can be directly bound via the HA and surface receptor to the red blood cell (or derivative). Virus purification from culture supernates and vaccine preparation can be achieved in a single step by the simple addition of the red blood cell (or derivative) to the supernate. A preferred technique for preparing viral antigens utilizes gamma irradiation which appears to favourably maintain the antigenicity of the preparation.

EP-A-0225329 and WO-A-9116073 contain specific examples of the preparation of the individual vaccine components.

In a fifth aspect, the present invention provides the use of at least one antigen derived from H. influenzae in the manufacture of an influenza vaccine. In one embodiment the influenza vaccine also includes at least one antigen derived from influenza virus.

In a further aspect, the invention provides a method for immunising a subject against lower respiratory tract infection, eg influenza, which comprises administering to the subject an effective amount of a vaccine or a vaccine composition of the invention.

Preferred features of each aspect of the invention are as for each other aspect mutatis mutandis.

The invention will now be described with reference to the following example. The example refers to the figures in which:

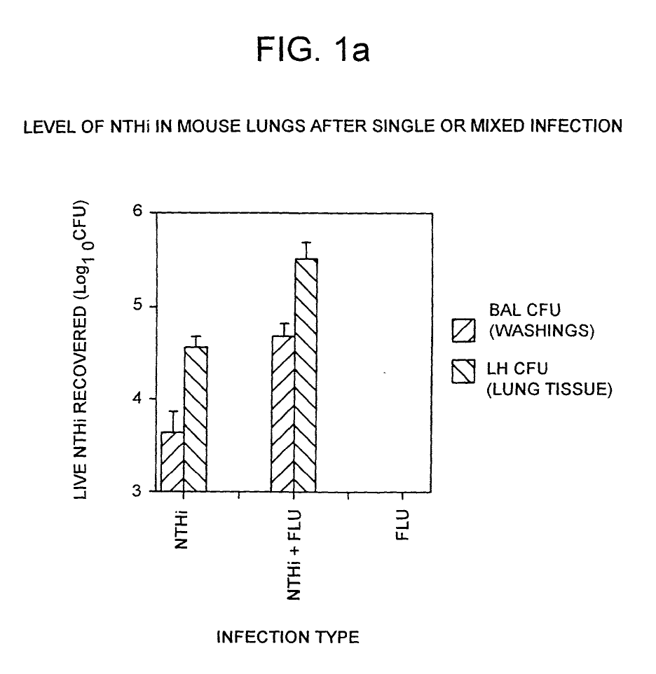

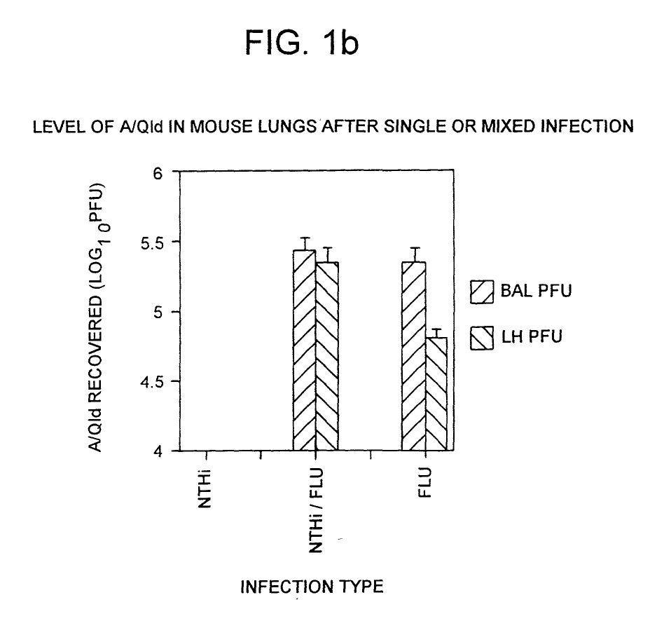

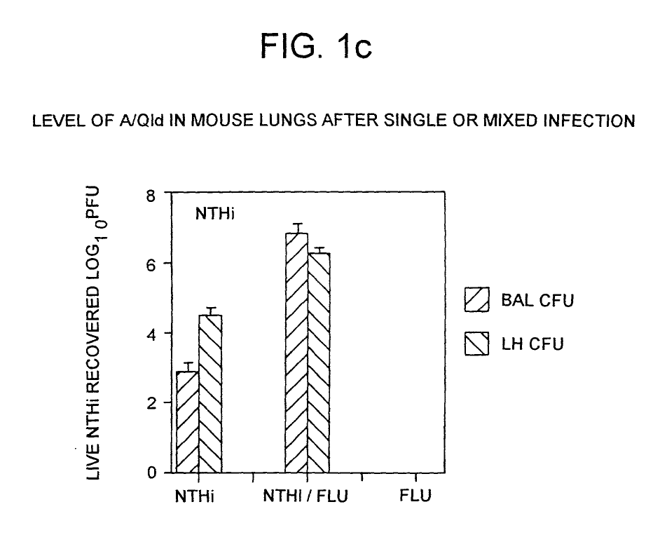

FIG. 1a: shows the level of NTHi in mouse lungs after single or mixed infection;

FIG. 1b: shows the level of A/QLD in mouse lungs after single or mixed infection;

FIG. 1c: shows the level of NTHi in mouse lungs after single or mixed infection; and

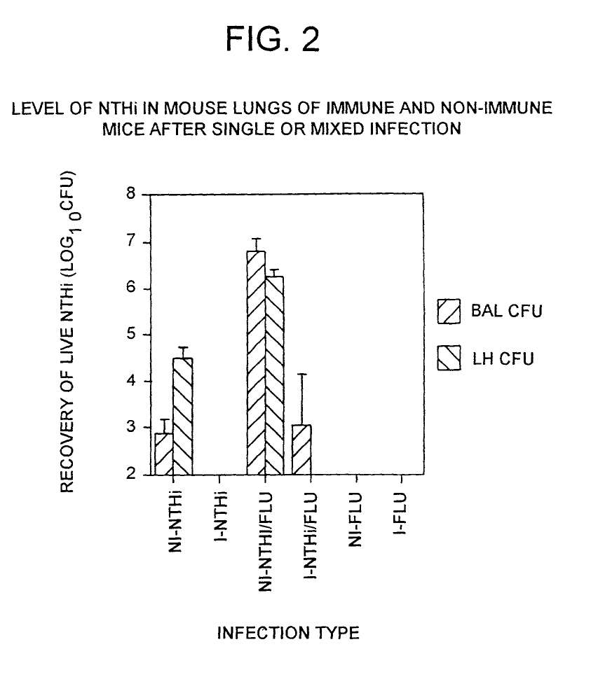

FIG. 2: shows the level of NTHi in mouse lungs of immune and non-immune mice after single or mixed infection.

### EXAMPLE 1

Enhanced infection in mixed NTHI/Influenza A/Qld respiratory infection of mice: Protection by pre-intestinal immunization with killed NTHI

Synergistic Infection in the Mouse Model

Balb/c mice (unimmunized were infected with Non-typeable Haemophilus influenzae (NTHi) or influenza virus (A/Qld) or a mixture of both NTHi and A/Qld. 24-48h after infection the mice were killed by pentobarbitone overdose and bronchoalveolar lavage fluid (BAL) was collected and the remaining lung tissue homogenized (LH). Both BAL and LH were subjected to determination of surviving NTHi or A/Qld. Compared to sole infection with either A/Qld or NTHi the mixed infection induces an enhanced bacterial (1 log, FIG. 1a; 2 logs, FIG. 1c) and viral (0.5 log in LH, FIG. 1b) infection.

Protection by Pre-Intetinal Immunization with Killed NTHi

Mice pre-immunized by intra-lumenal administration of killed NTHi followed by an intra-nasal boost with killed NTHi demonstrated protection against the increase in NTHi observed in the presence of A/Qld (FIG. 2).

We claim:

1. A vaccine comprising:
(i) at least one antigen obtained from H. influenzae; and
(ii) at least one antigen obtained from influenza virus.

2. The vaccine of claim 1 wherein the H. influenza antigen(s) is/are obtained from killed H. influenza bacteria.

3. The vaccine of claim 2 wherein at least one antigen from H. influenzae is provided in the form of whole killed H. influenza bacterial cells.

4. The vaccine of claim 1 wherein at least one antigen from H. influenzae is provided in isolated form.

5. The vaccine of claim 1 wherein at least one antigen from influenza virus is provided as inactivated whole influenza virus.

6. The vaccine of claim 1 wherein at least one antigen from influenza virus is provided as gamma irradiated influenza virus particles.

7. The vaccine of claim 3 wherein the vaccine contains between about 10⁹ to about 10¹³ bacteria.

8. The vaccine of claim 1 which is formulated for nasal administration.

9. The vaccine of claim 1 which is formulated for sub-lingual administration.

10. A vaccine comprising:
(i) at least one antigen obtained from killed H. inftuenzae; and
(ii) at least one antigen obtained from influenza virus.

11. The vaccine of claim 10 wherein the antigen obtained from influenza virus is from influenza virus surface-associated with red blood cells.

12. The vaccine of claim 12 wherein the antigen obtained from influenza virus is in the form of ghosts or membrane preparations.

13. The vaccine of claim 10 further comprising a pharmaceutical carrier.
The vaccine of claim 10 wherein the vaccine contains between about 10⁹ to about 10¹³ bacteria.

14. The vaccine of claim 10 which is formulated for nasal administration.

15. The vaccine of claim 10 which is formulated for sub-lingual administration.

16. A vaccine composition comprising:
(i) an enteral non-adjuvanted vaccine comprising killed H. influenzae and optionally a pharmaceutical carrier; and
(ii) a vaccine comprising at least one antigen obtained from influenza virus surface-associated with red blood cells or derivatives thereof.
